(19) United States
(12) Patent Application Publication
(10) Pub. No.: US 2013/0123613 A1
(43) Pub. Date: May 16, 2013

(54) Magnetic Particle Heating System And Method For Occlusion Of The Fallopian Tube

(71) Applicant: The Trustees of Dartmouth College, Hanover, NH (US)

(72) Inventors: B. Stuart Trembly, Hanover, NH (US); Paul D. Manganiello, Norwich, VT (US); P. Jack Hoopes, Hanover, NH (US)

(73) Assignee: THE TRUSTEES OF DARTMOUTH COLLEGE, Hanover, NH (US)

(21) Appl. No.: 13/717,527

(22) Filed: Dec. 17, 2012

Related U.S. Application Data
(63) Continuation of application No. PCT/US2011/040722, filed on Jun. 16, 2011.
(60) Provisional application No. 61/355,407, filed on Jun. 16, 2010.

(51) Int. Cl. A61F 6/20; A61B 6/12; A61B 5/01; A61B 8/08; A61B 1/303; A61M 31/00; A61N 2/06; A61B 6/00

(52) U.S. Cl. CPC A61F 6/20; A61N 2/06; A61B 6/12; A61B 6/485; A61B 8/0841; A61B 1/303; A61M 31/00; A61B 5/01
USPC 600/424; 600/12; 600/549

(57) ABSTRACT

In a magnetic particle heating method for occlusion of the Fallopian tubes, biocompatible magnetic particles are delivered transcervically to the entrance of the Fallopian tube. Placement of the particles (e.g., magnetic nanoparticles) in the Fallopian tube is confirmed, and a coil is positioned about or proximate the patient's abdomen. Alternating current is applied to the coil to couple magnetic energy into the nanoparticles, heating the nanoparticles and causing thermal injury to the Fallopian tube. Temperature within the Fallopian tube may be monitored during heating, to prevent overheating, or alternating current may be applied for a predetermined treatment time, with or without temperature monitoring. Each Fallopian tube may be treated with nanoparticles and the two tubes heated simultaneously, or the tubes may be sequentially treated and heated. Treatment may be visualized via in-office hysteroscope, fluoroscopy or ultrasound. Thermal injury to the Fallopian tube mucosa occludes the tube and completes sterilization.

### RELATED APPLICATIONS

This application is a continuation of PCT Patent Application PCT/US2011/040722, filed Jun. 16, 2011 which claims priority of U.S. Provisional Patent Application No. 61/355,407, filed Jun. 16, 2010 the disclosures of which are incorporated herein by reference.

### BACKGROUND

Tubal ligation is a conventional method of female sterilization in which a piece of the Fallopian tube is cut and sealed shut. The initial method of ligation and resection involved making an incision into the patient's abdomen, cutting the Fallopian tubes and tying the tubes off, blocking the passage of eggs to the uterus. Later innovations to the procedure included suturing the Fallopian tubes and using electrical current (electrocoagulation) to burn and destroy the Fallopian tube after cutting. More recently, laparoscopic clamping of the tube (with silicone rings, Hulka clips or other clips) has to a large extent replaced earlier surgical methods.

Two recent innovations to tubal sterilization include the Essure and Adiana methods. In the Adiana method, approved for use in the United States in 2009, radio frequency energy is used to remove cells lining a small area of the Fallopian tubes near the uterus, causing a lesion. A small implant is then inserted transcervically into the tubal opening and placed at the lesion site. Scar tissue grows into the implant to complete blockage of the tube.

In the Essure method, a coiled spring is inserted through the uterine cavity into each tubal opening (with a portion of the coil left inside the uterus) using a hysteroscope. The coil expands upon placement, to anchor into the Fallopian tube and to induce scarring. As the device becomes scarred in place, it forms a barrier to sperm and egg alike.

Although the Adiana and Essure methods eliminate surgical cutting—no incision or laparoscopic port is required, and the Fallopian tubes themselves are not cut—they are not without drawbacks. Placement of the implants may be time-consuming and cause significant discomfort to patients. Severe cramping and menstrual pain have been reported, and blockage is not always successful. In addition, improper placement of the implants may result in expulsion, and protrusion of the implant into the uterus may prevent later attempts at in vitro fertilization, should the woman change her mind about having children.

### SUMMARY

Magnetic hyperthermia is the name given to an experimental cancer treatment based upon the principle that magnetic nanoparticles, when subjected to an alternating magnetic field, produce heat. If magnetic nanoparticles are put inside a tumor and the patient is placed in an alternating magnetic field of a chosen amplitude and frequency, the tumor temperature increases. Temperatures above 45° C. may cause necrosis of the tumor cells, while temperatures of about 42° C. may improve the efficacy of chemotherapy treatment. See, e.g., U.S. Patent Application Publication Nos. 2003/0032995, 2003/0028071 and 2006/0269612 for additional description of magnetic hyperthermia as a palliative treatment.

In conventional practice of magnetic hyperthermia, great care is taken to avoid thermal injury to healthy tissue. Tumor-specific antigens may be used to coat magnetic nanoparticles, to insure that they bind with a tumor or tumors and not with healthy tissue.

The inventions disclosed herein advance the art of tubal sterilization by applying magnetic inductance hyperthermia to the Fallopian tubes. In particular, a method for tubal occlusion via magnetic particle heating is described below. The method utilizes magnetic particles to achieve permanent occlusion without requiring any implant. The method is therefore not limited by the need for correct implant placement, the time required to place the implant or the time required to confirm correct placement (or extra time required to correct improper placement). Likewise, because implants are not used, expulsion is not a concern.

It will be appreciated that although the magnetic particle heating system and methods disclosed herein are primarily described as utilizing nanoparticles, use of magnetic materials of other (i.e., larger) dimensions are also within the scope hereof.

In one embodiment, a method for occlusion of a Fallopian tube of a patient includes delivering biocompatible magnetic particles transcervically to the entrance of the Fallopian tube, and confirming placement of the particles in the Fallopian tube. A coil is positioned about or proximate the patient's abdomen, and alternating current is applied to the coil to couple magnetic energy into the particles, to heat the nanoparticles and cause thermal injury to the Fallopian tube.

In one embodiment, a system for administering magnetic particles to the Fallopian tube for heat-induced occlusion includes a catheter for transcervical insertion into the uterine ostium, and an injection device configured for fitting within the catheter. The injection device is loaded with magnetic particles that are (a) coated with a biocompatible material, and/or (b) suspended in a biocompatible carrier solution, for delivering the particles to the entrance of the Fallopian tube.

In one embodiment, a method for occlusion of a Fallopian tube of a patient includes visualizing the entrance of the Fallopian tube and delivering biocompatible magnetic particles transcervically to the entrance of the Fallopian tube. The method further includes positioning a coil about or proximate the patient's abdomen and applying alternating current to the coil to couple magnetic energy into the particles, to heat the particles and cause thermal injury to the Fallopian tube.

### BRIEF DESCRIPTION OF THE DRAWINGS

FIG. 1 depicts transcervical delivery of magnetic nanoparticles to the entrance of the Fallopian tube.

FIG. 2 is a partial view of FIG. 1, showing introduction of a temperature probe at the ostium to the intramural segment of the Fallopian tube.

FIG. 3 illustrates magnetic induction heating of the magnetic nanoparticles of FIGS. 1 and 2.

FIG. 4 is a partial view showing an occluding thermal injury to the Fallopian tube, caused by magnetic induction heating of the nanoparticles of FIGS. 1-4.

FIG. 5A shows exemplary placement of a coil about a patient's abdomen, to couple magnetic energy to the magnetic nanoparticles.

FIG. 5B shows exemplary placement of a coil above and proximate a patient's abdomen, to couple magnetic energy to the magnetic nanoparticles.

FIG. 6 is a flowchart illustrating a magnetic particle heating method for occlusion of the Fallopian tube, according to an embodiment.

FIG. 7 is a flowchart illustrating a magnetic particle heating method for occlusion of the Fallopian tube, according to an embodiment.

### DETAILED DESCRIPTION

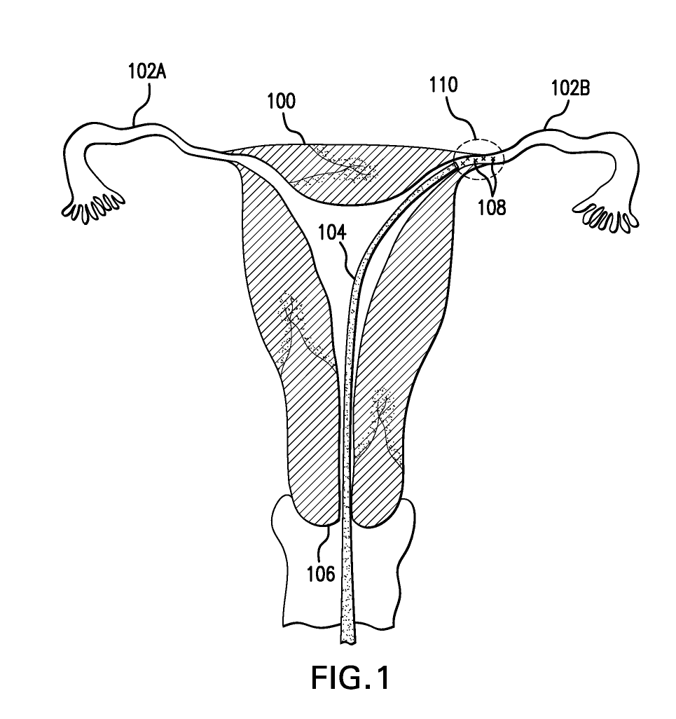

FIG. 1 is a schematic cross-sectional view of a uterus 100 and Fallopian tubes 102A and 102B, showing a catheter 104 inserted transcervically (through cervix 106) to deliver magnetic nanoparticles 108 (depicted by the letter “x”) to a target treatment area 110 of Fallopian tube 102B. Although nanoparticle 108 deposit is described herein with respect to injection/insertion into Fallopian tube 102B, it will be appreciated that the description below applies equally to deposit of nanoparticles 108 in Fallopian tube 102A.

Magnetic nanoparticles 108 may be iron oxide nanoparticles optionally coated with dextran, aminosilane or polyethylene glycol (PEG) for biocompatibility. Optionally, specific antibodies may be applied to nanoparticles 108 to direct nanoparticles 108 to the epithelial lining of Fallopian tube 102B/target area 110. If not coated with antibodies, nanoparticles 108 may be suspended in a gel-like matrix that coats Fallopian tube 102B mucosa (including mucosal crypts and folds) upon injection through the intramural portion of Fallopian tube 102B, at target area 110. Fibrin and/or a biocompatible adhesive may also be incorporated into the gel or carrier solution to aid hemostasis and/or tubal closure, and to keep nanoparticles 108 in place long enough for maximum absorption by cells of Fallopian tube 102B. Alternately or additionally, nanoparticles 108 may include magnetic materials having a Curie point equal to the intended treatment temperature, in addition to or as an alternative to iron oxide nanoparticles. Magnetic particles other than nanoparticles may also be used, where compatible with the treatment area.

Magnetic nanoparticles 108 have a viscosity that allows them to coat and directly heat (as explained below) the undulating Fallopian tube 102B lining/mucosal surface, permitting heat delivery (and damage) to a majority of the epithelial cells and superficial dermal cells lining Fallopian tube 102B, regardless of anatomical position of the cell (e.g., whether the cell is positioned at the tip of a fold or deep within a mucosal crypt). Nanoparticle 108 dosage is selected to achieve sufficient cell endocytosis of the particles to effect scarring and tubal occlusion. Effects of additional global heating, provided by particles or aggregates in the target treatment area 110 but not absorbed by Fallopian tube 102B cells, may also be considered when selecting nanoparticle dosage.

Insertion of catheter 104 and injection of nanoparticles 108 is for example performed under fluoroscopic, ultrasound or optical guidance, to confirm correct placement. Target area 110 is located at or near the entrance of Fallopian tube 102B, and is 2 cm or greater in length.

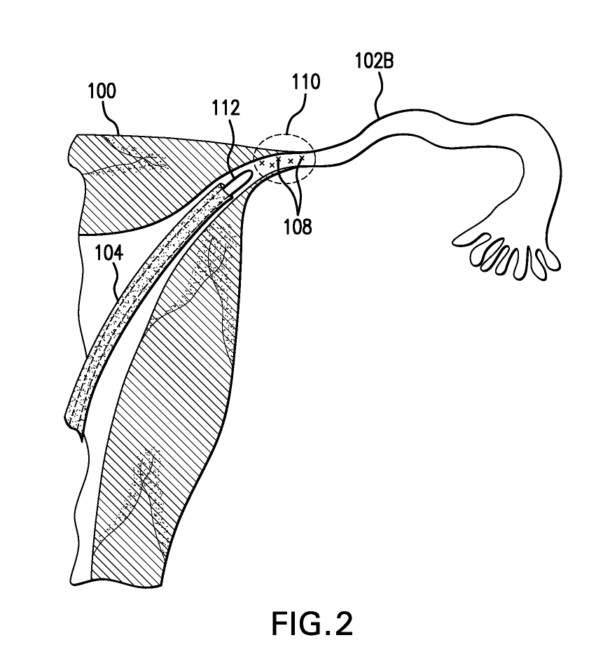

After injection of nanoparticles 108 through catheter 104, a temperature probe 112 is advanced through catheter 104 into the lumen of the uterine ostium, as shown in FIG. 2.

FIG. 2 is a partial, cross-sectional view of uterus 100 and Fallopian tube 102B, showing introduction of probe 112, which is for example a fiber optic probe. Temperature of the ostium to the intramural segment of the Fallopian tube (or entrance to the isthmus of Fallopian tube 102B) is monitored using probe 112 while a coil is energized to heat nanoparticles 108. Alternatively, some portion of the target region 110 is monitored by probe 112.

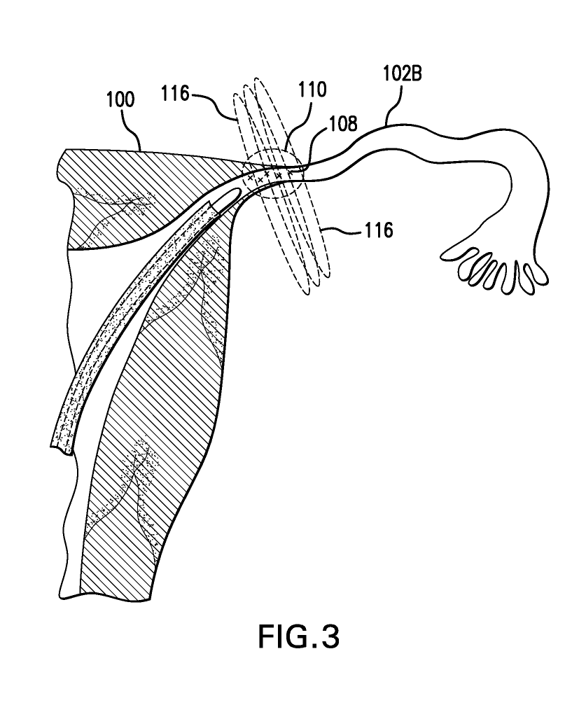

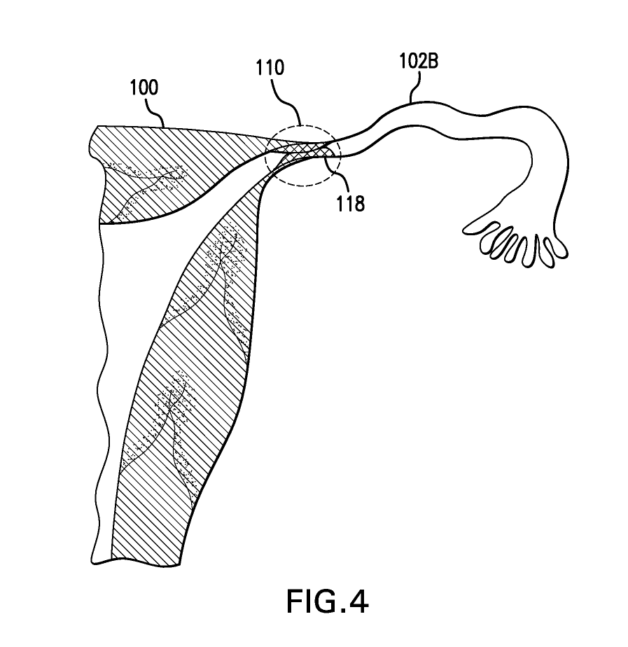

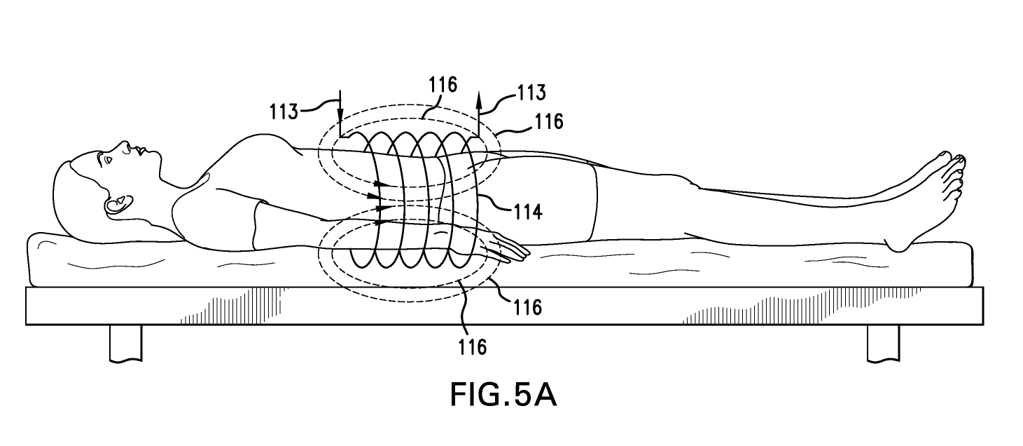

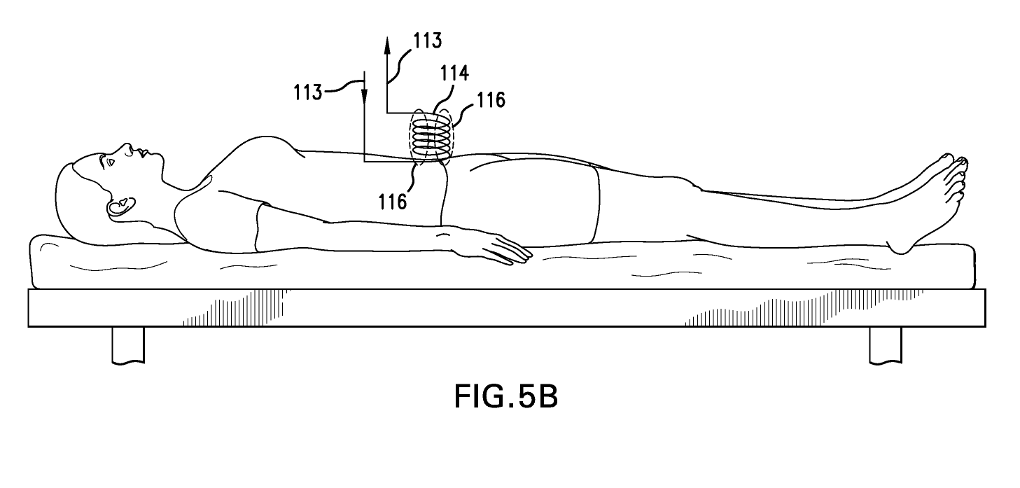

FIG. 3 schematically shows heating of nanoparticles 108 using alternating current. Alternating current (see current lines 113, FIGS. 5A and 5B) applied to a coil (not shown—see FIGS. 5A and 5B) induces a magnetic field manifested by magnetic lines of flux/field lines 116. Nanoparticles 108 interact with and become excited by the alternating magnetic field and are inductively heated to a temperature sufficient to cause an occluding thermal injury to Fallopian tube 102B. Target area 110 is heated to a pre-determined temperature value for a pre-determined time, for example, to 50° C. for ten minutes. Temperature is monitored by probe 112 to prevent overheating of uterus 100 and/or Fallopian tube 102B. Treatment results in a thermal lesion in Fallopian tube 102B mucosa and submucosa, which will occlude Fallopian tube 102B over most or all of target area 110, as illustratively shown in FIG. 4. The alternating magnetic field applied to the patient/nanoparticles 108 is selected and calculated to avoid induction of eddy current heating of abdominal tissue. In one alternate embodiment, nanoparticles 108 include magnetic materials with a Curie point equal to the treatment temperature. Alternately, magnetic materials having a Curie point equal to the treatment temperature may be used in place of nanoparticles 108. In another aspect, magnetic field strength, amount of nanoparticles 108 deposited (nanoparticle dosage) and treatment time may be predetermined values selected to effect tubal closure without harming neighboring, non-target tissue. In such aspect, internal temperature monitoring may not be necessary.

Following treatment, thermal lesion 118 (i.e., scar tissue) grows within Fallopian tube 102B to occlude the tube. After a healing period of about 10 days to 3 weeks, a hysterosalpingogram may be used to confirm tubal closure.

FIGS. 5A and 5B illustrate exemplary arrangements of coil 114 about and above a patient's abdomen (respectively). It will be appreciated that alternate configurations placing coil 114 about or near the abdomen and over Fallopian tubes 102A and 102B are within the scope hereof. It will also be appreciated that the treatment described herein for application to Fallopian tube 102B is also applied to tube 102A to complete occlusion and sterilization. FIG. 5A shows a coil 114 configuration surrounding the patient. Such a coil configuration is used to create MRI images. FIG. 5B illustrates an alternate configuration in which a single coil is placed above the patient and proximate the patient's abdomen.

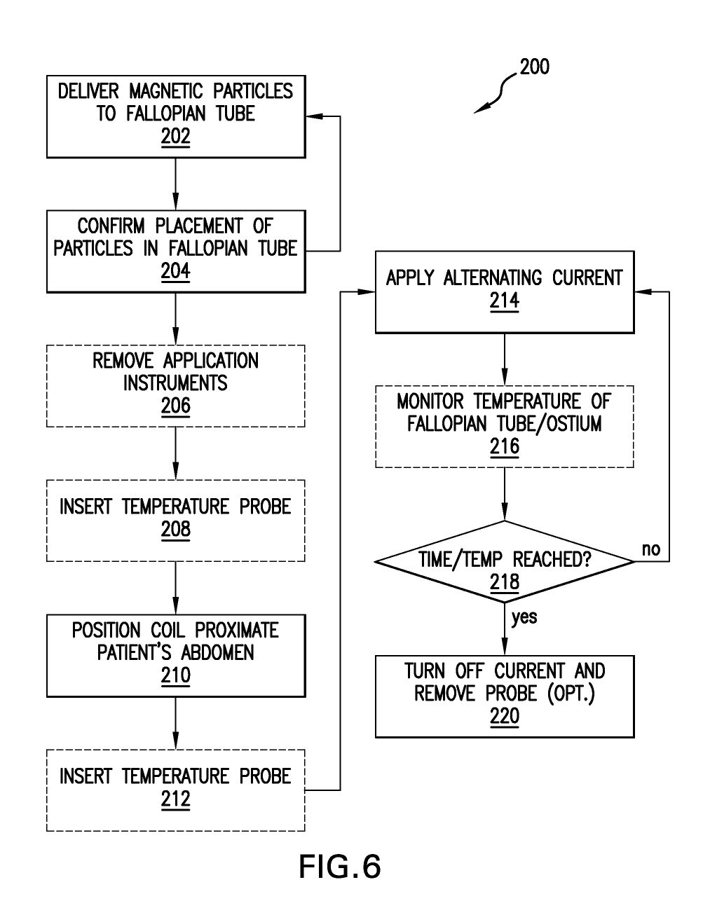

FIG. 6 shows a magnetic particle heating method 200 for occlusion of the Fallopian tubes. Magnetic particles are delivered to the Fallopian tube, in step 202, and correct placement of the particles confirmed, in step 204. Steps 202 and 204 preferably repeat for the second Fallopian tube, and optionally, until correct placement is confirmed. That is, nanoparticles are injected into the opening of each Fallopian tube prior to applying current. However, method 200 may alternately be carried out first on one Fallopian tube and then the other.

In step 206, application instruments are removed from the patient's body. A coil is positioned about or proximate the patient's abdomen in step 210. For example, the coil is positioned about the patient, as in FIG. 4A, or proximate the patient's abdomen over the Fallopian tubes, as in FIG. 4B. A temperature probe is optionally inserted to monitor temperature of the Fallopian tube and uterus, at step 208 or at step 212. Alternating current is applied to the coil, in step 214. A waiting period may be observed before current is applied to the coil, in order to allow uptake of the nanoparticles by the mucosal cells of the Fallopian tube. Allowing the Fallopian tube mucosa to take in the nanoparticles prior to heating may enhance energy absorption and cytotoxicity to the targeted cells.

Current applied to the coil, in step 214, induces a magnetic field that heats the magnetic particles. Temperature is optionally monitored within the Fallopian tube(s), in step 216, to prevent overheating. Alternately, frequency and intensity of the alternating magnetic field and the corresponding application time are preselected to effect tubal closure while preventing overheating of non-target tissue.

When the tube(s) are heated to a desired treatment temperature and/or for a desired amount of time (decision 218), the alternating current is turned off, in step 220. Any temperature monitoring probe, if used, is removed from the patient's body with step 220.

In one example of steps 202-220, magnetic nanoparticles 108 (alternately coated with biocompatible materials and/or suspended in a carrier solution/gel) are delivered through catheter 104 to target area 110 of Fallopian tube 102B. Method 200 may be performed under fluoroscopic, ultrasound or optical guidance, enabling the physician to confirm proper placement of nanoparticles 108 within target area 110, in step 204. Steps 202 and 204 repeat for Fallopian tube 102A. In one aspect, magnetic field strength and application time (and alternately, nanoparticle dosage) are preselected to achieve tubal closure without overheating surrounding tissues; in this case, catheter 104 is therefore removed from the patient's body in step 206, and internal temperature monitoring is not performed. Coil 114 is positioned about the patient's abdomen and/or proximate the Fallopian tubes, in step 210, and alternating current is applied to coil 114 in step 214 to induce an alternating magnetic field (See flux lines 116). Once the predetermined treatment time is reached (decision 218), current is turned off, in step 220.

In another aspect, temperature is internally monitored during treatment. Catheter 104 is not removed at step 206, and temperature probe 112 is advanced to the ostium to the intramural segment of Fallopian tube 102A and/or 102B via catheter 104 either at step 208, after placement of nanoparticles 108 is confirmed, or at step 212, just before alternating current is applied to coil 114.

Alternating current is applied to coil 114 in step 214, and temperature of Fallopian tube 102A, 102B and/or the uterine ostium of Fallopian tube 102A/102B are monitored with probe 112 during heating of nanoparticles 108, at step 216. Once target area 110 of one or both of Fallopian tubes 102A, 102B has been heated to a target treatment temperature for a predetermined length of time, current is turned off (i.e., power to coil 114 is turned off) and probe 112, catheter 104 and any additional instruments are removed from the patient's body, in step 220. It will be appreciated that in addition to temperature monitoring, visual monitoring, (for example, hysteroscopy, fluoroscopy or ultrasound) may also be used to confirm successful treatment.

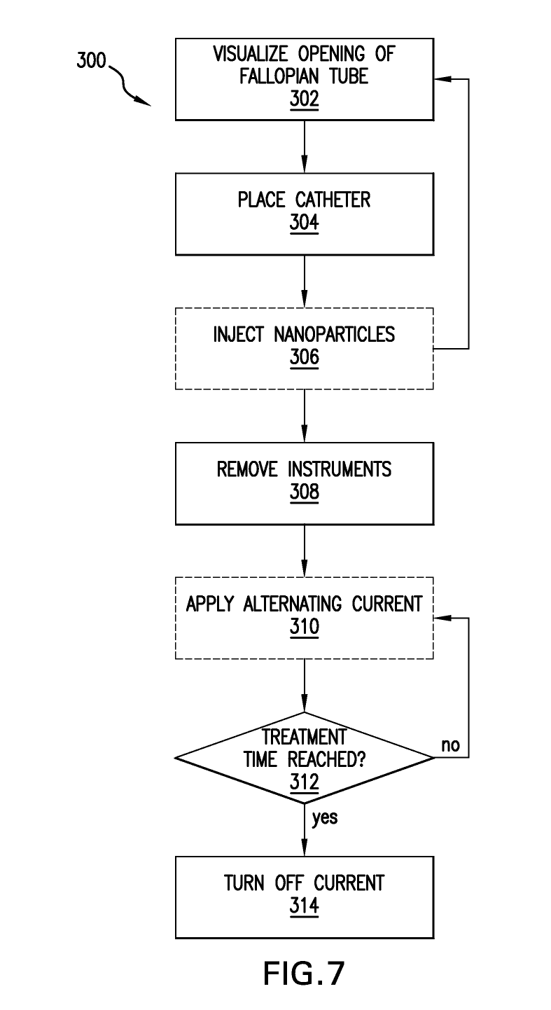

FIG. 7 shows a magnetic particle heating method 300 for occlusion of the Fallopian tubes. The opening of the Fallopian tube as it exits the uterine cavity is visualized in step 302. For example, a physician performs an in-office hysteroscopy, fluoroscopy or ultrasound to view the opening of the tube. An infusion catheter, e.g., catheter 104, is positioned at the mouth of the Fallopian tube in step 304, and nanoparticles are injected in step 306. Steps 304 and 306 are for example viewed using the hysteroscope. Steps 302-306 repeat for the second Fallopian tube. For example, the hysteroscope is re-positioned to visualize the opening of the second Fallopian tube, the catheter is re-positioned at the second tube opening and nanoparticles are injected.

Following treatment of the second Fallopian tube, delivery instruments are removed from the patient's body in step 308, and alternating current is applied in step 310. Once a predetermined treatment time is reached (decision 312), current is turned off and treatment ends, in step 314.

In one example of steps 302-314, hysteroscopy is used to visualize the opening of Fallopian tube 102B as it exits the uterine cavity. Catheter 104 is positioned at the mouth of Fallopian tube 102B, and nanoparticles 108 are injected at target area 110. Positioning and injection may be viewed using the hysteroscope. Following injection at target area 110, catheter 104 is positioned at the mouth of Fallopian tube 102A and nanoparticles 108 are injected, again, under hysteroscopy. Once both tubes 102A and 102B have been treated, instruments (e.g., catheter 104 and injection instruments) are removed from the patient's body, and alternating current is applied to heat nanoparticles 108 and surrounding tissue. Once a predetermined treatment time is reached, current is turned off and treatment ends.

As noted, nanoparticles 108 and any coating or carrier solution are biocompatible. Nanoparticles 108, and any coating or carrier solution, may also be biodegradable, such that the nanoparticles and accompanying material are absorbed and/or excreted by the body. Unlike conventional methods of female sterilization, the inventions described herein provide for permanent tubal occlusion without cutting into the patient's body, and without leaving behind any occluding implant, which might be expelled from the Fallopian tube. Costs associated with more invasive surgery and with implant devices themselves are therefore eliminated.

Also in contrast to conventional occluding implants, the occlusion method described herein may achieve tubal closure in 10 days to 3 weeks (based upon experimentation with magnetic nanoparticle tissue heating and an animal Fallopian tube model), whereas implant methods require 4-6 weeks of fibrosis to achieve tubal closure. Advantageously, the method described herein also requires minimal additional training, as it utilizes proven practices of minimally-invasive access to the Fallopian tube to deliver the nanoparticles and monitor temperature during treatment. Conventional imaging practices such as office hysteroscopy are used to confirm placement of the catheter and nanoparticles, after injection through the catheter. Fluoroscopy, ultrasound and other optical guidance techniques may also be employed to visualize the Fallopian tube opening and correctly place the nanoparticles. Hysterosalpingogram may be used to confirm tubal closure after a healing period following treatment.

While the present invention has been described above, it should be clear that changes and modifications may be made to the magnetic particle heating system and method for occlusion of the Fallopian tube without departing from the spirit and scope of this invention. For example, waiting periods may be instituted between steps as desired or necessary for successful treatment.

### Combinations of Features

Features described above as well as those claimed below may be combined in various ways without departing from the scope hereof. The following examples illustrate possible, non-limiting combinations:
(a) A method for occlusion of the Fallopian tube may include delivering biocompatible magnetic particles transcervically to the entrance of the Fallopian tube; confirming placement of the particles in the Fallopian tube; positioning a coil about or proximate the patient's abdomen, and applying alternating current to the coil to couple magnetic energy into the particles, to heat the particles and cause thermal injury to the Fallopian tube.
(b) In the method denoted as (a), placement may be confirmed by imaging the Fallopian tube entrance using office hysteroscopy, fluoroscopy or ultrasound.
(c) In the method denoted as (a) or (b), the particles may be or include iron oxide.
(d) In the method/s denoted as (a)-(c), the particles may include a biocompatible coating selected from the group of dextran, aminosilane, polyethylene glycol and antibodies.
(e) In the method denoted as (d), the biocompatible coating may include antibodies that are specific to the epithelial lining of the Fallopian tube.
(f) In the method/s denoted as (a)-(e), the particles may be suspended in a gel material.
(g) In the method denoted as (f), the gel material may include one or both of (a) fibrin, for enhancing hemostasis of the Fallopian tube and/or uterus after treatment, and (b) a biocompatible adhesive to enhance closure of the Fallopian tube after thermal injury.
(h) In the method/s denoted as (a)-(g), delivering the particles may include injecting the particles through a catheter.
(i) The method/s denoted as (a)-(h) may further include monitoring temperature within the Fallopian tube.
(j) In the method denoted as (i), monitoring temperature may include advancing a fiber optic temperature probe through the catheter and into the lumen of the Fallopian tube, to monitor thermal dose during heating.
(k) In the method/s denoted as (a)-(j), the magnetic particles may be magnetic nanoparticles.
(l) A system for administering particles to a Fallopian tube of a patient for heat-induced occlusion may include a catheter for transcervical insertion into the uterine ostium, and an injection device configured for fitting within the catheter and loaded with magnetic particles. The particles are coated with a biocompatible material and/or suspended in a biocompatible carrier solution, for delivering the particles to the entrance of the Fallopian tube.
(m) In the system denoted as (l), the particles may be or include iron oxide.
(n) In the system denoted as (l) or (m), the particles may be coated with a material selected from the group of dextran, aminosilane, polyethylene glycol and antibodies specific to the epithelial lining of the Fallopian tube.
(o) In the system/s denoted as (l)-(n), the particles may be suspended in a carrier solution including fibrin, to enhance hemostasis after treatment of the Fallopian tube.
(p) In the system/s denoted as (l)-(o), the particles may be suspended in a carrier solution including a biocompatible adhesive, to enhance closure of the Fallopian tube.
(q) In the system/s denoted as (l)-(p), the particles may be magnetic nanoparticles, micron-sized magnetic materials or millimeter-sized magnetic materials.
(r) A method for occlusion of the Fallopian tubes may include visualizing the entrance of the Fallopian tube; delivering biocompatible magnetic particles transcervically to the entrance of the Fallopian tube; positioning a coil about or proximate the patient's abdomen, and applying alternating current to the coil to couple magnetic energy into the particles, to heat the particles and cause thermal injury to the Fallopian tube.
(s) In the method denoted as (r), delivering biocompatible magnetic particles may include placing a catheter end at the entrance of the Fallopian tube and injecting the particles through the catheter to the entrance of the Fallopian tube.
(t) In the method denoted as (r) or (s), visualizing the entrance of the Fallopian tube may include using a hysteroscope.
(u) The method/s denoted as (r)-(t) may further include determining and employing one or more of a particle dosage, a magnetic field strength and a treatment time to achieve the thermal injury to the Fallopian tube without overheating non-target tissue.
(v) The method/s denoted as (r)-(u) may further include utilizing delivery instruments to deliver the biocompatible magnetic particles, and removing the delivery instruments from the patient's body before applying the alternating current.
(w) In the method/s denoted as (r)-(v), the magnetic particles may be magnetic nanoparticles.

What is claimed is:

1. A method for occlusion of a Fallopian tube of a patient, comprising:
delivering biocompatible magnetic particles transcervically to the Fallopian tube;
confirming placement of the particles in the Fallopian tube;
positioning a coil about or proximate the patient's abdomen; and
applying alternating current to the coil to couple magnetic energy into the particles, to heat the particles and cause thermal injury to the Fallopian tube.

2. The method of claim 1, wherein confirming placement comprises imaging the Fallopian tube entrance using office hysteroscopy, fluoroscopy or ultrasound.

3. The method of claim 1, the particles comprising iron oxide.

4. The method of claim 3, wherein the magnetic particles comprise magnetic nanoparticles.

5. The method of claim 1, the particles comprising a biocompatible coating selected from the group of dextran, aminosilane, polyethylene glycol and antibodies specific to the epithelial lining of the Fallopian tube.

6. The method of claim 1, the particles being suspended in a gel material.

7. The method of claim 6, the gel material including one or both of (a) fibrin, and (b) a biocompatible adhesive.

8. The method of claim 6, wherein the magnetic particles comprise magnetic nanoparticles.

9. The method of claim 1, wherein delivering the particles comprises injecting the particles through a catheter.

10. The method of claim 1, further comprising monitoring temperature within the Fallopian tube.

11. The method of claim 10, wherein delivering the particles comprises injecting the particles through a catheter, and wherein monitoring temperature comprises advancing a temperature probe through the catheter and into the lumen of the Fallopian tube.

12. The method according to any of claim 10, the magnetic particles comprising magnetic nanoparticles.

13. A system for administering particles to the Fallopian tube for heat-induced occlusion, comprising:
a catheter for transcervical insertion into the uterine ostium; and
an injection device configured for fitting within the catheter and loaded with magnetic particles (a) coated with a biocompatible material and/or (b) suspended in a biocompatible carrier solution, for delivering the magnetic particles to the Fallopian tube.

14. A system of claim 13, the particles comprising iron oxide.

15. A system of claim 14, the particles being coated with a material selected from the group of dextran, aminosilane, polyethylene glycol and antibodies specific to the epithelial lining of the Fallopian tube.

16. A system of claim 13 wherein the carrier solution is a gel.

17. A system of claim 16, the particles comprising magnetic nanoparticles.

18. A system of claim 13, the particles suspended in a carrier solution including fibrin.

19. A system of claim 13, the particles suspended in a carrier solution including a biocompatible adhesive, the adhesive adapted to enhance closure of the Fallopian tube.

20. A method for occlusion of the Fallopian tubes, comprising:
visualizing the entrance of the Fallopian tube;
delivering biocompatible magnetic particles transcervically to the entrance of the Fallopian tube;
positioning a coil about or proximate the patient's abdomen; and
applying alternating current to the coil to couple magnetic energy into the particles, to heat the particles and cause thermal injury to the Fallopian tube.

21. The method of claim 20, wherein delivering biocompatible magnetic particles comprises placing a catheter end at the entrance of the Fallopian tube and injecting the particles through the catheter to the entrance of the Fallopian tube.

22. The method of claim 20, wherein visualizing the entrance of the Fallopian tube comprises using a hysteroscope.

23. The method of claim 20 further comprising determining and employing one or more of a magnetic particle dosage, a magnetic field strength and a treatment time to achieve the thermal injury to the Fallopian tube without overheating non-target tissue.

24. The method according to claim 23, the magnetic particles comprising magnetic nanoparticles.

25. The method of claim 20, wherein delivering biocompatible magnetic particles comprises utilizing delivery instruments, and further comprising removing the delivery instruments from the patient's body before applying the alternating current.

26. The method according to claim 25, the magnetic particles comprising magnetic nanoparticles.

27. The method of claim 20, the particles being suspended in a gel material comprising one or both of (a) fibrin, and (b) a biocompatible adhesive.

28. The method of claim 27, wherein the magnetic particles comprise magnetic nanoparticles.
